(19) United States
(12) Patent Application Publication
(10) Pub. No.: US 2011/0315139 A1
(43) Pub. Date: Dec. 29, 2011

(54) AUTOMATIC FRESH GAS CONTROL SYSTEM

(75) Inventor: James Nyal Mashak, Madison, WI (US)

(73) Assignee: General Electric Company, Schenectady, NY (US)

(21) Appl. No.: 12/821,331

(22) Filed: Jun. 23, 2010

(51) Int. Cl. A61M 16/01

(52) U.S. Cl. 128/203.14

(57) ABSTRACT

An anesthesia system is disclosed herein. The anesthesia system includes a pneumatic circuit comprising an inspiratory limb, an expiratory limb, and a sensor. The anesthesia system also includes an anesthesia machine comprising a controller. The controller is operatively connected to the sensor, and is configured to identify a respiratory phase of the patient based on feedback from the sensor. The controller is further configured to regulate the flow rate of a fresh gas in response to the identified respiratory phase.

### FIELD OF THE INVENTION

This disclosure relates generally to a system configured to automatically regulate the flow of fresh gas during manual ventilation.

### BACKGROUND OF THE INVENTION

In general, medical ventilators systems are used to provide respiratory support to patients undergoing anesthesia and respiratory treatment whenever the patient's ability to breath is compromised. The primary function of the medical ventilator system is to maintain suitable pressure and flow of gases inspired and expired by the patient. Medical ventilator systems often include a manual system comprising a collapsible reservoir configured to allow a clinician to deliver manual breaths to the patient.

The manual system is implemented to ventilate a patient by repeatedly compressing and releasing the collapsible reservoir. When the collapsible reservoir is compressed, inhalation gas is transferred to the patient. When the collapsible reservoir is subsequently released, the patient passively exhales due to the lungs' elasticity. Fresh gas is generally continuously introduced into the system. An adjustable pressure limit (APL) valve is traditionally provided to limit the pressure level in the manual system and thereby regulate the volume of inhalation gas transferred to the patient during each compression of the collapsible reservoir.

One problem with conventional medical ventilator systems relates to potential for accumulation of fresh gas. More precisely, during intervals between collapsible reservoir compression, fresh gas introduced into the system can accumulate and generate increased pressure. This increased pressure can impede exhalation and must be manually bled off using the APL valve, which can create a distraction and represents an inefficient use of resources.

### BRIEF DESCRIPTION OF THE INVENTION

The above-mentioned shortcomings, disadvantages and problems are addressed herein which will be understood by reading and understanding the following specification.

In an embodiment, an anesthesia system includes a pneumatic circuit comprising an inspiratory limb adapted to deliver an inspiratory gas to a patient, an expiratory limb adapted to deliver an expiratory gas from the patient, and a sensor in pneumatic communication with either the inspiratory limb or the expiratory limb. The anesthesia system also includes an anesthesia machine pneumatically coupled with the pneumatic circuit. The anesthesia machine includes a controller operatively connected to the sensor. The controller is configured to identify a respiratory phase of the patient based on feedback from the sensor, and to regulate the flow rate of a fresh gas in response to the identified respiratory phase.

In another embodiment, an anesthesia system includes a pneumatic circuit comprising an inspiratory limb, an expiratory limb, a first flow sensor, and a second flow sensor. The anesthesia system also includes a collapsible reservoir and an anesthesia machine that are pneumatically coupled with the pneumatic circuit. The anesthesia machine includes a controller configured to identify an interval between collapsible reservoir compressions based on feedback from the first and second flow sensors, and to regulate the flow rate of a fresh gas such that a pressure level within the pneumatic circuit does not exceed a predetermined target pressure level during the identified interval.

Various other features, objects, and advantages of the invention will be made apparent to those skilled in the art from the accompanying drawings and detailed description thereof.

### BRIEF DESCRIPTION OF THE DRAWINGS

FIG. 1 is a schematic representation of an anesthesia system in accordance with an embodiment; and

FIG. 2 is a schematic representation of a pneumatic circuit of the anesthesia system of FIG. 1 in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

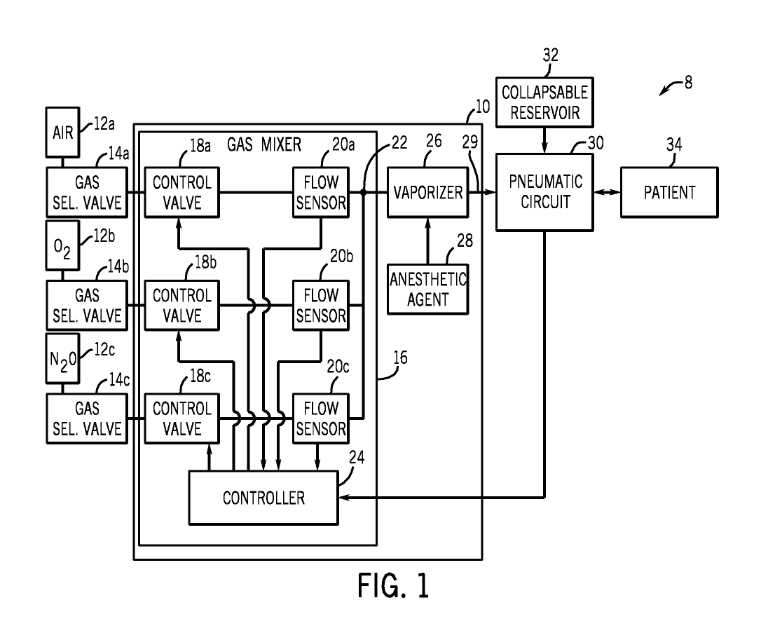

Referring to FIG. 1, an anesthesia system 8 is schematically depicted in accordance with an embodiment. The anesthesia system 8 includes an anesthesia machine 10, a plurality of gas storage devices 12a, 12b and 12c, a plurality of gas selector valves 14a, 14b, and 14c, a pneumatic circuit 30, and a collapsible reservoir or breathing bag 32. The anesthesia machine 10 is shown for illustrative purposes and it should be appreciated that other types of anesthesia machines may alternately be implemented. In a typical hospital environment, the gas storage devices 12a, 12b and 12c are centrally located storage tanks configured to supply medical gas to multiple anesthesia machines and multiple hospital rooms. The storage tanks are generally pressurized to facilitate the transfer of the medical gas to the anesthesia machine 10.

The gas storage devices 12a, 12b and 12c will hereinafter be described as including an air tank 12a, an oxygen (O2) tank 12b, and a nitrous oxide (N2O) tank 12c, respectively, however it should be appreciated that other storage devices and other types of gas may alternatively be implemented. The gas storage tanks 12a, 12b and 12c are each connected to one of the gas selector valves 14a, 14b, and 14c, respectively. The gas selector valves 14a, 14b and 14c may be implemented to shut off the flow of medical gas from the storage tanks 12a, 12b and 12c when the anesthesia machine 10 is not operational. When one of the gas selector valves 14a, 14b and 14c is opened, gas from a respective storage tank 12a, 12b and 12c is transferred under pressure to the anesthesia machine 10.

The anesthesia machine 10 includes a gas mixer 16 adapted to receive medical gas from the storage tanks 12a, 12b and 12c. The gas mixer 16 includes a plurality of control valves 18a, 18b and 18c that are respectively connected to one of the gas selector valves 14a, 14b and 14c. The gas mixer 16 also includes a plurality of flow sensors 20a, 20b and 20c that are each disposed downstream from a respective control valve 18a, 18b, and 18c. After passing through one of the control valves 18a, 18b and 18c, and passing by one of the flow sensors 20a, 20b and 20c, the individual gasses (i.e., air, O2 and N2O) are combined to form a mixed gas at the mixed gas outlet 22.

The control valves 18a, 18b and 18c and the flow sensors 20a, 20b and 20c are each connected to a controller 24. The controller 24 is configured to operate the control valves 18a, 18b and 18c in a response to gas flow rate feedback from the sensors 20a, 20b and 20c. Accordingly, the controller 24 can be implemented to maintain a selectable flow rate for each gas (i.e., air, O2 and N2O) such that the mixed gas at the mixed gas outlet 22 comprises a selectable ratio of air, O2 and N2O. The mixed gas flows to a vaporizer 26 where an anesthetic agent 28 may be vaporized and added to the mixed gas from the mixed gas outlet 22. The anesthetic agent 28 and/or mixed gas combination is referred to as inhalation gas or fresh gas 29, which passes through the pneumatic circuit 30 and is delivered to the patient 34.

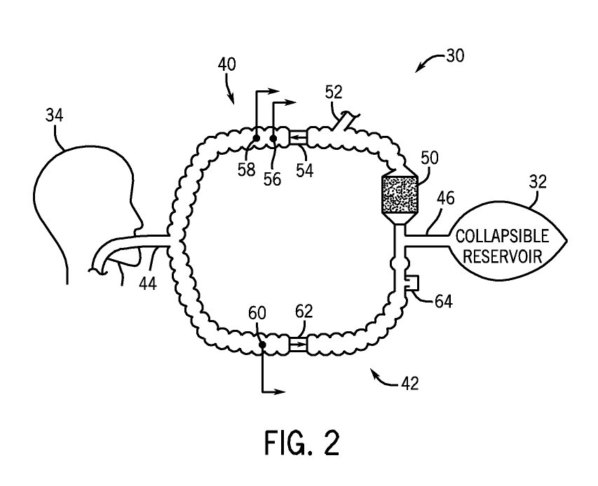

As will be described in detail hereinafter, the pneumatic circuit 30 is configured to facilitate the transfer of fresh gas 29 from the anesthesia machine 10 to the patient 34, and to vent exhalation gas from the patient 34 to a hospital scavenging system (not shown). The pneumatic circuit 30 is also configured to generate a feedback signal from one or more of the sensors 56, 58 and/or 60 (shown in FIG. 2) that is transmittable to the controller 24 for purposes of regulating fresh gas 29 flow rate. The collapsible reservoir 32 may be manually compressed to transfer fresh gas 29 to the patient 34 in a known manner.

Referring to FIG. 2, an exemplary embodiment of the pneumatic circuit 30 is shown in more detail. The pneumatic circuit 30 may include an inspiratory channel or limb 40, an expiratory channel or limb 42, a Y-piece 44 and a T-piece 46. The Y-piece 44 pneumatically couples the patient 34 with the inspiratory limb 40 and the expiratory limb 42. The T-piece 46 pneumatically couples the collapsible reservoir 32 with the inspiratory limb 40 and the expiratory limb 42.

The inspiratory limb 40 comprises one or more tubes configured to direct fresh gas 29 and/or recycled exhalation gas to the patient 34. The inspiratory limb 40 may include a CO2 absorber 50, a fresh gas inlet 52, a one-way valve 54, a pressure sensor 56, and a flow sensor 58.

The CO2 absorber 50 is adapted to remove CO2 from the patient's exhalation gas to produce recycled exhalation gas. The recycled exhalation gas is transferable back to the patient 34 to reuse and thereby conserve anesthetic agent 28. The fresh gas inlet 52 is pneumatically coupled with and adapted to receive fresh gas 29 from the anesthesia machine 10. The one-way valve 54 is adapted to regulate fluid flow through the inspiratory limb 40 such that fluid is only transferable in a direction toward the patient 34. For purposes of this disclosure, the term fluid should be defined to include any substance that continually deforms or flows under an applied shear stress such as, for example, a liquid or a gas. The pressure sensor 56 and flow sensor 58 are respectively configured to measure the pressure and flow rate of a fluid passing through the inspiratory limb 40, and to transfer measurement data to the controller 24 (shown in FIG. 1). The pressure and flow sensors 56, 58 may comprise known technology and therefore will not be described in detail.

The expiratory limb 42 comprises one or more tubes configured to direct exhalation gas from the patient 34. The exhalation gas from the patient 34 can be passed through the CO2 absorber 50 to produce recycled exhalation gas that is transferable back to the patient 34 for rebreathing. Alternatively, some or all of the exhalation gas from the patient 34 can be vented to atmosphere or passed through a hospital scavenging system. The expiratory limb 42 may include a flow sensor 60, a one-way valve 62, and an adjustable pressure limit (APL) valve 64.

The flow sensor 60 is configured to measure the flow rate of a fluid passing through the expiratory limb 42, and to transfer measurement data to the controller 24 (shown in FIG. 1). The one-way valve 62 is adapted to regulate fluid flow through the expiratory limb 42 such that fluid is only transferable in a direction away from the patient 34. The APL valve 64 is adapted to set an upper pressure limit within the pneumatic circuit 30.

It should be appreciated that in conventional systems fresh gas can accumulate within a pneumatic circuit during intervals between collapsible reservoir compression. As the fresh gas accumulates, the pressure within the pneumatic circuit approaches the limit set by the APL valve thereby rendering patient exhalation more difficult. The accumulated fresh gas is generally bled off by manually adjusting the APL valve pressure limit. Manually bleeding off fresh gas accumulation is distracting and represents an inefficient use of valuable resources, and is also potentially wasteful of anesthetic agent 28 that may be vented to atmosphere or passed through a scavenging system.

Referring to FIGS. 1 and 2, the system 8 is adapted to automatically regulate the accumulation of fresh gas as will now be described in detail. According to one embodiment, the controller 24 may be configured to regulate the flow of fresh gas 29 from the anesthesia machine 10 based on feedback from one or more of the pressure sensor 56, the flow sensor 58 and the flow sensor 60 such that pressure within the pneumatic circuit 30 is automatically maintained at or near a predefined target pressure.

The predefined target pressure is generally at least that which is necessary to maintain a minimum amount of gas in the collapsible reservoir 32 so that any subsequent compression will have the desired effect of transferring inhalation gas to the patient 34. It has been observed that 1 cm H2O is minimally sufficient to inflate the collapsible reservoir 32. In instances in which it is desirable to maintain a positive end expiratory pressure (PEEP), the predefined target pressure is set to the prescribed PEEP level.

The following will provide several non-limiting examples of how the controller 24 may be configured to regulate the flow of fresh gas 29 from the anesthesia machine 10 such that pressure within the pneumatic circuit 30 is automatically maintained at or near a predefined target pressure.

According to a first embodiment the controller 24 may be configured to identify a patient's respiration phase. Thereafter, the controller 24 can regulate the flow rate of fresh gas 29 based on the identified respiration phase such that the target pressure level is maintained within the pneumatic circuit 30. For purposes of this disclosure, the term respiration phase should be defined to include inhalation, exhalation and intervals between breaths. It should be appreciated that the identification of respiration phase advantageously may be implemented to regulate anesthesia machine operation during assisted breathing and/or spontaneous breathing.

The patient's respiration phase can be identified in the following non-limiting manner. The controller 24 can identify patient inhalation based on an increase in pressure measured by the pressure sensor 56, and/or a measured fluid flow from the flow sensor 58. The controller 24 can identify patient exhalation based on a decrease in pressure measured by the pressure sensor 56, and/or a measured fluid flow from the flow sensor 60. The controller 24 can identify intervals between patient breaths, or correspondingly intervals between compressible reservoir 32 compression, based on the absence of a measured fluid flow from the sensors 58, 60.

During inhalation, the controller 24 can reduce the flow rate of fresh gas 29 to allow for the increase in pressure attributable to collapsible reservoir 32 compression while generally maintaining the target pressure level within the pneumatic circuit 30. During intervals between patient breaths (or collapsible reservoir 32 compression), the controller 24 can regulate the flow rate of fresh gas 29 based on the measured pressure level from the pressure sensor 56. More precisely, the controller 24 can increase fresh gas flow rate if the measured pressure level is below the target pressure level, and can reduce the fresh gas flow rate if the measured pressure level is as at or above the target pressure level.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

I claim:

1. An anesthesia system comprising:
a pneumatic circuit comprising:
an inspiratory limb adapted to deliver an inspiratory gas to a patient;
an expiratory limb adapted to deliver an expiratory gas from the patient; and
a sensor in pneumatic communication with one of the inspiratory limb and the expiratory limb; and
an anesthesia machine pneumatically coupled with the pneumatic circuit, said anesthesia machine comprising a controller operatively connected to the sensor, said controller configured to identify a respiratory phase of the patient based on feedback from the sensor, and to regulate the flow rate of a fresh gas in response to the identified respiratory phase.

2. The anesthesia system of claim 1, wherein the controller is configured to reduce the flow rate of a fresh gas when a patient inhalation is identified.

3. The anesthesia system of claim 1, wherein the controller is configured to regulate the flow rate of a fresh gas such that a pressure level within the pneumatic circuit does not exceed a predetermined target pressure level during an interval between patient breaths.

4. The anesthesia system of claim 1, wherein the controller is configured to regulate the flow rate of a fresh gas to inflate a collapsible reservoir.

5. The anesthesia system of claim 1, wherein the controller is configured to regulate the flow rate of a fresh gas to maintain a positive end expiratory pressure level.

6. The anesthesia system of claim 1, wherein the sensor comprises a pressure sensor in pneumatic communication with the inspiratory limb.

7. The anesthesia system of claim 1, wherein the sensor comprises a pressure sensor in pneumatic communication with the inspiratory limb; a first flow sensor in pneumatic communication with the inspiratory limb; and a second flow sensor pneumatic communication with the expiratory limb.

8. The anesthesia system of claim 1, wherein the pneumatic circuit comprises an adjustable pressure limit valve.

9. The anesthesia system of claim 1, wherein the pneumatic circuit comprises a first one-way valve disposed within the inspiratory limb and a second one-way valve disposed within the expiratory limb.

10. An anesthesia system comprising:
a pneumatic circuit comprising:
an inspiratory limb adapted to deliver an inspiratory gas to a patient;
an expiratory limb adapted to deliver an expiratory gas from the patient;
a first flow sensor in pneumatic communication with the inspiratory limb; and
a second flow sensor in pneumatic communication with the expiratory limb;
a collapsible reservoir pneumatically coupled with the pneumatic circuit; and
an anesthesia machine pneumatically coupled with the pneumatic circuit, said anesthesia machine comprising a controller configured to identify an interval between collapsible reservoir compressions based on feedback from the first and second flow sensors, and to regulate the flow rate of a fresh gas such that a pressure level within the pneumatic circuit does not exceed a predetermined target pressure level during the identified interval.

11. The anesthesia system of claim 10, wherein the pneumatic circuit further comprises a pressure sensor in pneumatic communication with the inspiratory limb.

12. The anesthesia system of claim 11, wherein the controller is configured to regulate the flow rate of the fresh gas based on feedback from the pressure sensor.

13. The anesthesia system of claim 10, wherein the pneumatic circuit comprises an adjustable pressure limit valve.

14. The anesthesia system of claim 10, wherein the pneumatic circuit comprises a first one-way valve disposed within the inspiratory limb and a second one-way valve disposed within the expiratory limb.
